(19) United States
(12) Patent Application Publication
(10) Pub. No.: US 2016/0345871 A1
(43) Pub. Date: Dec. 1, 2016

(54) INFORMATION PROCESSING DEVICE, INFORMATION PROCESSING METHOD, AND PROGRAM

(71) Applicant: NORITSU PRECISION CO., LTD., Wakayama-shi, Wakayama (JP)

(72) Inventors: Shuichi Matsumoto, Wakayama-shi (JP); Takeshi Murai, Wakayama-shi (JP); Masayoshi Uetsuji, Wakayama-shi (JP)

(73) Assignee: NORITSU PRECISION CO., LTD., Wakayama-shi, Wakayama (JP)

(21) Appl. No.: 15/116,422

(22) PCT Filed: Jan. 22, 2015
(86) PCT No.: PCT/JP2015/051631

§ 371 Date: Aug. 3, 2016

(30) Foreign Application Priority Data
Feb. 7, 2014 (JP) 2014-021822

(51) Int. Cl. A61B 5/11; G06K 9/00; G06T 7/00; A61B 5/00; A61B 5/16

(52) U.S. Cl. CPC A61B 5/1128; A61B 5/0077; A61B 5/1115; A61B 5/1123; A61B 5/16; A61B 5/746; A61B 5/747; A61B 5/7405; A61B 5/0075; G06T 7/0044; G06K 9/00335; G06T 7/0051; A61B 2562/0219; A61B 2576/00; G06T 2207/20144

(57) ABSTRACT

The present invention provides an information processing device that acquires a captured image including depth information that is captured by an image capturing device including a depth sensor, and determines whether a positional relationship between a person being watched over and a region of a bed satisfies a predetermined condition, based on the depth for each pixel. In the case where a state in which the depth for each pixel within the captured image cannot be acquired by the depth sensor continues for a given period of time or longer, the information processing device notifies that there is a possibility that watching over of the person being watched over is not being performed normally.

### TECHNICAL FIELD

The present invention relates to an information processing device, an information processing method, and a program.

### BACKGROUND ART

There is a technology that judges an in-bed event and an out-of-bed event, by respectively detecting human body movement from a floor region to a bed region and detecting human body movement from the bed region to the floor region, passing through a boundary edge of an image captured diagonally downward from an upward position inside a room (Patent Literature 1).

Also, there is a technology that sets a watching region for determining that a patient who is sleeping in bed has carried out a getting up action to a region directly above the bed that includes the patient who is in bed, and judges that the patient has carried out the getting up action, in the case where a variable indicating the size of an image region that the patient is thought to occupy in the watching region of a captured image that includes the watching region from a lateral direction of the bed is less than an initial value indicating the size of an image region that the patient is thought to occupy in the watching region of a captured image obtained from a camera in a state in which the patient is sleeping in bed (Patent Literature 2).

### CITATION LIST

### Patent Literature

Patent Literature 1: JP 2002-230533A

Patent Literature 2: JP 2011-005171A

### SUMMARY OF INVENTION

### Technical Problem

In recent years, accidents involving people who are being watched over such as inpatients, facility residents and care-receivers rolling or falling from bed, and accidents caused by the wandering of dementia patients have tended to increase year by year. As a method of preventing such accidents, watching systems, such as illustrated in Patent Literatures 1 and 2, for example, that detect the behavior of a person who is being watched over, such as sitting up, edge sitting and being out of bed, by capturing the person being watched over with an image capturing device (camera) installed in the room and analyzing the captured image have been developed.

In such watching systems, when there is a change in the environment in which watching over is performed (hereinafter, also referred to as the “watching environment”), it may no longer be possible to appropriately detect the behavior of the person being watched over. For example, due to a change in the orientation of the image capturing device, the person being watched over may no longer appear in the captured image, and it may no longer be possible to detect the behavior of the person being watched over. When the watching system is left in such a state, a situation in which watching over of the person being watched over cannot be performed normally will be perpetuated.

The present invention was, in one aspect, made in consideration of such points, and it is an object thereof to provide a technology that makes it possible to prevent a watching system from being left in a state in which watched over of a person being watched over can no longer be performed normally.

### Solution to Problem

The present invention employs the following configurations in order to solve the abovementioned problem.

That is, an information processing device according to one aspect of the present invention includes an image acquisition unit configured to acquire a captured image captured by an image capturing device that is installed in order to watch for behavior, in a bed, of a person being watched over and includes a depth sensor for measuring a depth of a subject, the captured image including depth information indicating the depth for each pixel within the captured image that is measured by the depth sensor, a behavior detection unit configured to detect behavior, related to the bed, of the person being watched over, by determining whether a positional relationship within real space between the person being watched over and a region of the bed satisfies a predetermined condition, based on the depth for each pixel within the captured image that is indicated by the depth information, an anomaly determination unit configured to determine whether a state in which the depth for each pixel within the captured image cannot be acquired by the depth sensor has continued for a given period of time or longer, and a notification unit configured to perform, in a case where it is determined that the state in which the depth for each pixel within the captured image cannot be acquired by the depth sensor has continued for a given period of time or longer, notification for informing that there is a possibility that watching over of the person being watched over is not being performed normally.

In view of this, the information processing device according to the above configuration determines whether the positional relationship between a reference plane of the bed and the person being watched over in the height direction of the bed within real space satisfies a predetermined condition, based on the depth for each pixel within the captured image. The information processing device according to the above configuration then infers the positional relationship within real space between the person being watched over and the bed, based on the result of this determination, and detects behavior of the person being watched over that is related to the bed.

In other words, the information processing device according to the above configuration detects the behavior of the person being watched over, based on depth information included in the captured image. Hence, in a state such as where this depth information cannot be acquired, the information processing device according to the above configuration can no longer detect the behavior of the person being watched over. In order to address this, the information processing device according to the above configuration determines whether the state in which the depth for each pixel within the captured image cannot be acquired by the depth sensor included in the image capturing device has continued for a given period of time or longer. In the case where it is determined that the state in which the depth for each pixel within the captured image cannot be acquired by the depth sensor has continued for a given period of time or longer, the information processing device according to the above configuration performs notification for informing that there is a possibility that watching over of the person being watched over is not being performed normally.

The information processing device according to the above configuration is thereby able to report to a user or the like that there is a possibility that watching over cannot be performed normally, in the case where it is determined that the state in which the depth for each pixel within a captured image cannot be acquired by the depth sensor has continued for a given period of time or longer. Thus, the watching system can be prevented from being left in a state in which there is a problem with the watching over that occurs due to not being able to acquire depth information. That is, according to the above configuration, the watching system can be prevented from being left in a state in which watching over of the person being watched over cannot be performed normally.

Note that the person being watched over is a person whose behavior in bed is watched over using the present invention, such as an inpatient, a facility resident or a care-receiver, for example. On the other hand, the person who watches over the person being watched over is a nurse, a facility staff member or a care-provider, for example. Also, behavior related to the bed refers to behavior that the person being watched over may possibly carry out at the place where his or her bed is located, and includes, for example, sitting up in bed, edge sitting on the bed, being over the bed rails, falling from bed, and being out of bed. Here, edge sitting refers to a state in which the person being watched over is sitting on the edge of the bed. Also, being over the rails refers to a state in which the person being watched over is leaning out over rails of the bed.

Also, as another mode of the information processing device according to the above aspect, the anomaly determination unit may determine, in a case where the depth cannot be acquired for more than a predetermined proportion of the region of the bed, that the state in which the depth for each pixel within the captured image cannot be acquired by the depth sensor has occurred. With this configuration, the information processing device determines that a state in which the depth for each pixel within a captured image cannot be acquired has occurred, in the case where the depth cannot be acquired for the region of the bed that serves as a reference for the behavior of the person being watched over. Thus, it is possible to prevent it being determined that a state in which the depth for each pixel within a captured image cannot be acquired has occurred, in the case where the depth cannot be acquired for a region unrelated to detecting the behavior of the person being watched over. According to this configuration, the possibility of the occurrence of an anomaly in the watching system being erroneously reported can thereby be reduced.

Also, as another mode of the information processing device according to the above aspect, the information processing device may further include a foreground extraction unit configured to extract a foreground region of the captured image from a difference between the captured image and a background image set as a background of the captured image. Also, the behavior detection unit may detect the behavior, related to the bed, of the person being watched over, by determining whether the positional relationship within real space between the person being watched over and the region of the bed satisfies a predetermined condition, utilizing, as a position of the person being watched over, a position within real space of a target appearing in the foreground region that is specified based on the depth for each pixel within the foreground region.

With this configuration, a foreground region of the captured image is specified by extracting the difference between a background image and the captured image. This foreground region is a region in which change has occurred from the background image. Thus, the foreground region includes, as an image related to the person being watched over, a region in which change has occurred due to movement of the person being watched over, or in other words, a region in which there exists a part of the body of the person being watched over that has moved (hereinafter, also referred to as the “moving part”). Therefore, by referring to the depth for each pixel within the foreground region that is indicated by the depth information, it is possible to specify the position of the moving part of the person being watched over within real space.

In view of this, the information processing device according to the above configuration determines whether the positional relationship between the reference plane of the bed and the person being watched over satisfies a predetermined condition, utilizing the position within real space of a target appearing in the foreground region that is specified based on the depth for each pixel within the foreground region as the position of the person being watched over. That is, the predetermined condition for detecting the behavior of the person being watched over is set assuming that the foreground region is related to the behavior of the person being watched over. The information processing device according to the above configuration detects the behavior of the person being watched over, based on the height at which the moving part of the person being watched over exists with respect to the reference plan of the bed within real space.

Here, the foreground region can be extracted with the difference between the background image and the captured image, and can thus be specified without using advanced image processing. Thus, according to the above configuration, it becomes possible to detect the behavior of the person being watched over with a simple method.

Also, as another mode of the information processing device according to the above aspect, the image capturing device may further include an acceleration sensor, the anomaly determination unit may determine, after detecting an impact to the image capturing device based on the acceleration sensor, whether a shift of a given amount or more has occurred in an image capturing range of the image capturing device, by comparing the captured image prior to the impact and the captured image after the impact, and the notification unit may perform, in a case where it is determined that a shift of a given amount or more has occurred in the image capturing range of the image capturing device, notification for informing that there is a possibility that watching over of the person being watched over is not being performed normally.

As described above, due to a change in the orientation of the image capturing device, the person being watched over may no longer appear in the captured image, and it may no longer be possible to detect the behavior of the person being watched over. According to this configuration, it can be reported to a user or the like that there is a possibility that watching over cannot be performed normally, in the case where it is determined that a shift of a given amount or more has occurred in an image capturing range of the image capturing device. Thus, the watching system can be prevented from being left in a state in which there is a problem with the watching over that occurs due to a change in the orientation of the image capturing device.

Also, as another mode of the information processing device according to the above aspect, the information processing device is connected to a nurse call system for calling a person who watches over the person being watched over. Also, the notification unit may perform a call by the nurse call system, as the notification for informing that there is a possibility that watching over of the person being watched over is not being performed normally. According to this configuration, the occurrence of an anomaly in the watching system can be reported through a nurse call system.

Also, as another mode of the information processing device according to the above aspect, the information processing device may be connected to an audio output device for outputting audio. Also, the notification unit may cause, in a case where the call by the nurse call system cannot be performed, the audio output device to perform output of predetermined audio, as the notification for informing that there is a possibility that watching over of the person being watched over is not being performed normally, instead of the call by the nurse call system. According to this configuration, the occurrence of an anomaly in the watching system can be reported, even in the case where calling of the nurse call system cannot be performed normally.

Also, as another mode of the information processing device according to the above aspect, the information processing device may be connected to a display device that is for performing screen display. Also, the notification unit may cause, in a case where the call by the nurse call system cannot be performed, the display device to perform screen display in a predetermined mode, as the notification for informing that there is a possibility that watching over of the person being watched over is not being performed normally, instead of the call by the nurse call system. According to this configuration, the occurrence of an anomaly in the watching system can be reported, even in the case where calling of the nurse call system cannot be performed normally.

Also, as another mode of the information processing device according to the above aspect, the anomaly determination unit may determine whether the information processing device recognizes the image capturing device, and the notification unit may perform, in a case where it is determined that the information processing device does not recognize the image capturing device, notification for informing that there is a possibility that watching over of the person being watched over is not being performed normally.

When the image capturing device can no longer be recognized, the information processing device can no longer acquire the captured image that is utilized in order to detect the behavior of the person being watched over, and thus watching over of the person being watched over can no longer be executed normally. According to this configuration, since notification of the occurrence of an anomaly in the watching system is performed in such a state, the watching system can be prevented from being left in a state in which there is a problem with the watching over that occurs due to no longer being able to acquire the captured image.

Also, as another mode of the information processing device according to the above aspect, the anomaly determination unit may determine whether detection of the behavior of the person being watched over by the behavior detection unit has not been executed for a given period of time or longer, and the notification unit may perform, in a case where it is determined that detection of the behavior of the person being watched over by the behavior detection unit has not been executed for a given period of time or longer, notification for informing that there is a possibility that watching over of the person being watched over is not being performed normally. In the case where detection of the behavior of the person being watched over is not executed, there is a possibility that watching over of the person being watched over is not being performed normally. According to this configuration, since the occurrence of an anomaly is reported in such a case, the watching system can be prevented from being left in a state in which there is a problem with the watching over that occurs due to detection of the behavior of the person being watched over not being executed for a given period of time or longer.

Also, as another mode of the information processing device according to the above aspect, the depth sensor that is included in the image capturing device may be an infrared depth sensor that measures depth based on infrared irradiation. With an infrared depth sensor, the depth of a subject can be acquired even in dark places. Thus, according to this configuration, it becomes possible to acquire the depth of a subject without being affected by the brightness of the place where watching over of the person being watched over is performed, and to detect the behavior of the person being watched over.

Note that as another mode of the information processing device according to each of the above modes, the present invention may be an information processing system, an information processing method, or a program that realizes each of the above configurations, or may be a storage medium having such a program recorded thereon and readable by a computer or other apparatus, machine or the like. Here, a storage medium that is readable by a computer or the like is a medium that stores information such as programs by an electrical, magnetic, optical, mechanical or chemical action. Also, the information processing system may be realized by one or a plurality of information processing devices.

For example, an information processing method according to one aspect of the present invention is an information processing method in which a computer executes a step of acquiring a captured image captured by an image capturing device that is installed in order to watch for behavior, in a bed, of a person being watched over and includes a depth sensor for measuring a depth of a subject, the captured image including depth information indicating the depth for each pixel within the captured image that is measured by the depth sensor, a step of detecting behavior, related to the bed, of the person being watched over, by determining whether a positional relationship within real space between the person being watched over and a region of the bed satisfies a predetermined condition, based on the depth for each pixel within the captured image that is indicated by the depth information, a step of determining whether a state in which the depth for each pixel within the captured image cannot be acquired by the depth sensor has continued for a given period of time or longer, and a step of performing, in a case where it is determined that the state in which the depth for each pixel within the captured image cannot be acquired by the depth sensor has continued for a given period of time or longer, notification for informing that there is a possibility that watching over of the person being watched over is not being performed normally.

Also, a program according to one aspect of the present invention, for example, is a program for causing a computer to execute a step of acquiring a captured image captured by an image capturing device that is installed in order to watch for behavior, in a bed, of a person being watched over and includes a depth sensor for measuring a depth of a subject, the captured image including depth information indicating the depth for each pixel within the captured image that is measured by the depth sensor, a step of detecting behavior, related to the bed, of the person being watched over, by determining whether a positional relationship within real space between the person being watched over and a region of the bed satisfies a predetermined condition, based on the depth for each pixel within the captured image that is indicated by the depth information, a step of determining whether a state in which the depth for each pixel within the captured image cannot be acquired by the depth sensor has continued for a given period of time or longer, and a step of performing, in a case where it is determined that the state in which the depth for each pixel within the captured image cannot be acquired by the depth sensor has continued for a given period of time or longer, notification for informing that there is a possibility that watching over of the person being watched over is not being performed normally.

### Advantageous Effects of Invention

According to the present invention, it becomes possible to prevent a watching system from being left in a state in which watching over of a person who is being watched over can no longer be performed normally.

### BRIEF DESCRIPTION OF DRAWINGS

FIG. 1 shows an example of a situation in which the present invention is applied.

FIG. 2 shows an example of a captured image in which a gray value of each pixel is determined according to the depth for that pixel.

FIG. 3 illustrates a hardware configuration of an information processing device according to an embodiment.

FIG. 4 illustrates depth according to the embodiment.

FIG. 5 illustrates a functional configuration according to the embodiment.

FIG. 6 illustrates a processing procedure by the information processing device when detecting the behavior of a person being watched over in the embodiment.

FIG. 7 illustrates a screen that is displayed at the time of the information processing device according to the embodiment performing watching over of the person being watched over.

FIG. 8 illustrates the three-dimensional distribution of a subject in an image capturing range that is specified based on depth information that is included in a captured image.

FIG. 9 illustrates the three-dimensional distribution of a foreground region that is extracted from a captured image.

FIG. 10 schematically illustrates a detection region for a watching system according to the embodiment to detect sitting up.

FIG. 11 schematically illustrates a detection region for the watching system according to the embodiment to detect being out of bed.

FIG. 12 schematically illustrates a detection region for the watching system according to the embodiment to detect edge sitting.

FIG. 13 illustrates a processing procedure for preventing the watching system from being left in a state in which watching, which is executed by the information processing device according to the embodiment, cannot be performed normally.

FIG. 14 illustrates the relationship between dispersion and the degree of spread of a region.

### DESCRIPTION OF EMBODIMENTS

Hereinafter, an embodiment (hereinafter, also described as “the present embodiment”) according to one aspect of the present invention will be described based on the drawings. The present embodiment described below is, however, to be considered in all respects as illustrative of the present invention. It is to be understood that various improvements and modifications can be made without departing from the scope of the present invention. In other words, in implementing the present invention, specific configurations that depend on the embodiment may be employed as appropriate.

Note that data appearing in the present embodiment will be described using natural language, and will, more specifically, be designated with computer-recognizable quasi-language, commands, parameters, machine language, and the like.

### 1. Exemplary Application Situation

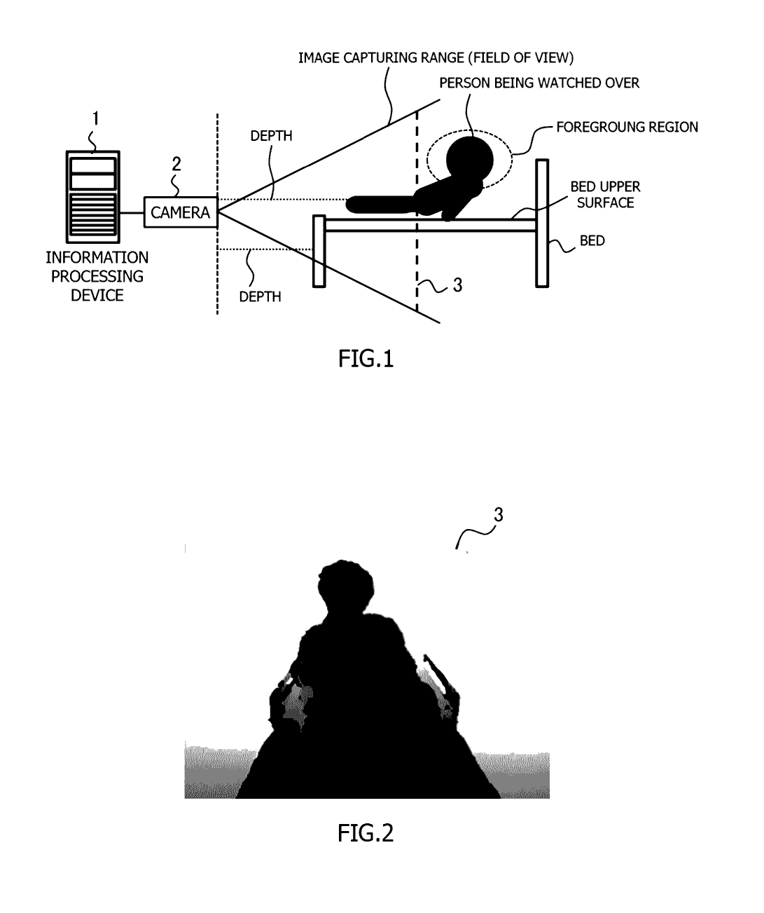

First, a situation to which the present invention is applied will be described using FIG. 1. FIG. 1 schematically shows an example of a situation to which the present invention is applied. In the present embodiment, a situation in which the behavior of an inpatient or a facility resident is watched over in a medical facility or a nursing facility is assumed as an illustration of a person being watched over. The person who watches over the person being watched over (hereinafter, also referred to as the “user”) is a nurse or a facility staff member, for example. Watching over of the behavior in bed of a person being watched over is performed, utilizing a watching system that includes an information processing device 1 and a camera 2.

The watching system according to the present embodiment acquires a captured image 3 in which the person being watched over and the bed appear, by capturing the behavior of the person being watched over using the camera 2. The watching system then detects the behavior of the person being watched over and watches over the behavior of the person being watched over, by using the information processing device 1 to analyze the captured image 3 that is acquired with the camera 2.

The camera 2 corresponds to an image capturing device of the present invention, and is installed in order to watch over the behavior in bed of the person being watched over. The position in which the camera 2 can be installed is not particularly limited, and, in the present embodiment, the camera 2 is installed forward of the bed in the longitudinal direction. FIG. 1 illustrates a situation in which the camera 2 is viewed from the side, and the up-down direction in FIG. 1 corresponds to the height direction of the bed. Also, the left-right direction in FIG. 1 corresponds to the longitudinal direction of the bed, and the direction perpendicular to the page in FIG. 1 corresponds to the width direction of the bed.

The camera 2 according to the present embodiment includes a depth sensor (depth sensor 8 discussed later) for measuring the depth of a subject, and is able to acquire a depth corresponding to each pixel within a captured image. Thus, the captured image 3 that is acquired by this camera 2 includes depth information indicating the depth that is obtained for every pixel, as illustrated in FIG. 1.

The data format of the captured image 3 including this depth information is not particularly limited, and may be selected, as appropriate, according to the embodiment. The captured image 3 may be data indicating the depth of a subject within the image capturing range, or may be data in which the depth of a subject within the image capturing range is distributed two-dimensionally (e.g., depth map), for example. Also, the captured image 3 may include an RGB image together with depth information. Furthermore, the captured image 3 may be a moving image or may be a static image.

FIG. 2 shows an example of such a captured image 3. The captured image 3 illustrated in FIG. 2 is an image in which the gray value of each pixel is determined according to the depth for that pixel. Blacker pixels indicate decreased distance to the camera 2. On the other hand, whiter pixels indicate increased distance to the camera 2. This depth information enables the position within real space (three-dimensional space) of the subject within the image capturing range to be specified.

More specifically, the depth of a subject is acquired with respect to the surface of that subject. The position within real space of the surface of the subject captured on the camera 2 can then be specified, by using the depth information that is included in the captured image 3. In the present embodiment, the captured image 3 captured by the camera 2 is transmitted to the information processing device 1. The information processing device 1 then infers the behavior of the person being watched over, based on the acquired captured image 3.

The information processing device 1 according to the present embodiment specifies a foreground region within the captured image 3, by extracting the difference between the captured image 3 and a background image that is set as the background of the captured image 3, in order to infer the behavior of the person being watched over based on the captured image 3 that is acquired. The foreground region that is specified is a region in which change has occurred from the background image, and thus includes the region in which the moving part of the person being watched over exists. In view of this, the information processing device 1 detects the behavior of the person being watched over, utilizing the foreground region as an image related to the person being watched over.

For example, in the case where the person being watched over sits up in bed, the region in which the part relating to the sitting up (upper body in FIG. 1) appears is extracted as the foreground region, as illustrated in FIG. 1. It is possible to specify the position of the moving part of the person being watched over within real space, by referring to the depth for each pixel within the foreground region that is thus extracted.

It is possible to infer the behavior in bed of the person being watched over based on the positional relationship between the moving part that is thus specified and the bed. For example, in the case where the moving part of the person being watched over is detected upward of the upper surface of the bed, as illustrated in FIG. 1, it can be inferred that the person being watched over has carried out the movement of sitting up in bed. Also, in the case where the moving part of the person being watched over is detected in proximity to the side of the bed, for example, it can be inferred that the person being watched over is moving to an edge sitting state.

In view of this, the information processing device 1 utilizes the position within real space of a target appearing in the foreground region that is specified based on the depth for each pixel within the foreground region as the position of the person being watched over. Specifically, the information processing device 1 according to the present embodiment detects the behavior of the person being watched over, based on the positional relationship within real space between the target appearing in the foreground region and the bed. In other words, the information processing device 1 detects the behavior of the person being watched over, based on where, within real space, the moving part of the person being watched over is positioned with respect to the bed. Thus, when depth information can no longer be acquired or the image capturing range changes, for example, due to the watching environment changing, it may no longer be possible for the behavior of the person being watched over to be detected normally. When the watching system is left in such a state, a state in which watching over of the person being watched over cannot be performed normally will be perpetuated.

In order to address this, the information processing device 1 according to the present embodiment determines whether the state in which the depth for each pixel within the captured image 3 cannot be acquired by the depth sensor that is included in the camera 2 has continued for a given period of time or longer. In the case where it is determined that the state in which the depth for each pixel within the captured image 3 cannot be acquired by the depth sensor has continued for a given period of time or longer, the information processing device 1 according to the present embodiment performs a notification for informing that there is a possibility that watching over of the person being watched over is not being performed normally.

The information processing device 1 according to the present embodiment is thereby able to report to a user or the like that there is a possibility that watching over cannot be performed normally, in the case where it is determined that the state in which the depth for each pixel within the captured image 3 cannot be acquired by the depth sensor has continued for a given period of time or longer. Thus, according to the present embodiment, the watching system can be prevented from being left in a state in which there is a problem with the watching over that occurs due to not being able to acquire depth information. That is, the watching system can be prevented from being left in a state in which watching over of the person being watched over can no longer be performed normally.

### 2. Exemplary Configuration

### Hardware Configuration

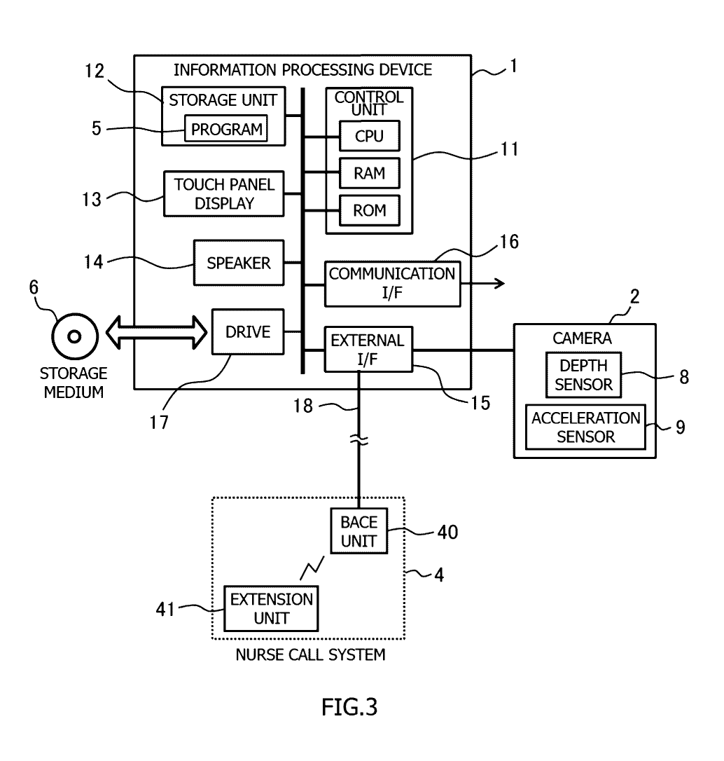

Next, the hardware configuration of the information processing device 1 will be described using FIG. 3. FIG. 3 illustrates the hardware configuration of the information processing device 1 according to the present embodiment. The information processing device 1 is a computer in which a control unit 11 including a CPU, a RAM (Random Access Memory), a ROM (Read Only Memory) and the like, a storage unit 12 storing information such as a program 5 that is executed by the control unit 11, a touch panel display 13 for performing image display and input, a speaker 14 for outputting audio, an external interface 15 for connecting to an external device, a communication interface 16 for performing communication via a network, and a drive 17 for reading programs stored in a storage medium 6 are electrically connected, as illustrated in FIG. 3. In FIG. 3, the communication interface and the external interface are respectively described as a “communication I/F” and an “external I/F”.

Note that, in relationship to the specific hardware configuration of the information processing device 1, constituent elements can be omitted, replaced or added, as appropriate, according to the embodiment. For example, the control unit 11 may include a plurality of processors. Also, for example, the touch panel display 13 may be replaced by an input device and a display device that are respectively separately connected independently. Also, for example, the speaker 14 may be omitted. Also, for example, the speaker 14 may be connected to the information processing device 1 as an external device, rather than as an internal device of the information processing device 1. Also, the information processing device 1 may incorporate the camera 2.

The information processing device 1 may be provided with a plurality of external interfaces 15, and may be connected to a plurality of external devices. In the present embodiment, the information processing device 1 is connected to the camera 2 via the external interface 15. The camera 2 according to the present embodiment is installed in order to watch over the behavior in bed of the person being watched over. This camera 2 is provided with a depth sensor 8 for measuring the depth of a subject, and an acceleration sensor 9 for measuring the movement of the camera 2. The type and measurement method of the depth sensor 8 and the acceleration sensor 9 may be selected as appropriate according to the embodiment. For example, a TOF (Time Of Flight) sensor or the like can be given as the depth sensor 8. Also, a capacitance detection sensor, a piezoresistance sensor, a heat detection sensor and the like can be given as types of the acceleration sensor 9.

Note that the place (e.g., ward of a medical facility) where watching over of the person being watched over is performed is a place where the bed of the person being watched over is located, or in other words, the place where the person being watched over sleeps. Thus, watching over of the person being watched over may possibly be performed in a dark place.

In view of this, in order to acquire the depth without being affected by the brightness of the place where image capture is performed, an infrared depth sensor that measures depth based on infrared irradiation is preferably used as the depth sensor 8. Kinect by Microsoft Corporation, Xtion by Asus and Carmine by PrimeSense can be given as comparatively cost-effective image capturing devices that include such an infrared depth sensor.

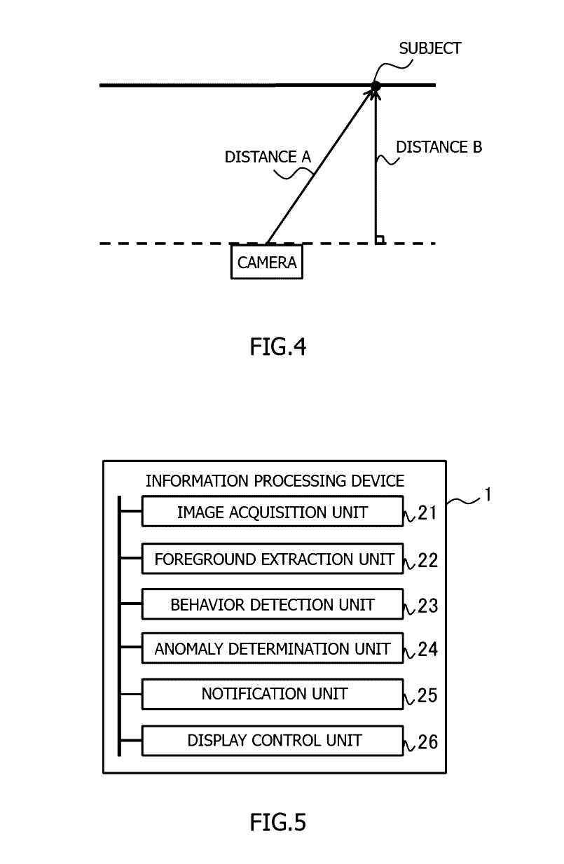

Here, the depth measured by a depth sensor according to the present embodiment will be described in detail using FIG. 4. FIG. 4 shows an example of the distances that can be treated as a depth according to the present embodiment. This depth represents the depth of a subject. As illustrated in FIG. 4, the depth of the subject may be represented in a distance A of a straight line between the camera and the object, or may be represented in a distance B of a perpendicular down from the horizontal axis of the camera with respect to the subject, for example. That is, the depth according to the present embodiment may be the distance A or may be the distance B. In the present embodiment, the distance B will be treated as the depth. However, the distance A and the distance B are exchangeable with each other using Pythagorean theorem or the like, for example. Thus, the following description using the distance B can be directly applied to the distance A.

Also, the information processing device 1 is, as illustrated in FIG. 3, connected to a nurse call system 4 via the external interface 15. The hardware configuration and functional configuration of the nurse call system 4 may be selected, as appropriate, according to the embodiment. The nurse call system 4 is a device for calling a user (nurse, facility staff member, etc.) who is watching over the person being watched over, and a known device may be used as the nurse call system. The nurse call system 4 according to the present embodiment is provided with a base unit 40 that is connected to the information processing device 1 with wiring 18, and an extension unit 41 capable of wireless communication with this base unit 40.

The base unit 40 is installed in the place where a user is stationed, for example. The base unit 40 is mainly utilized in order to call the user in the station. On the other hand, the extension unit 41 is generally carried around by a user. The extension unit 41 is utilized in order to call the user who is carrying around the extension unit 41. The base unit 40 and the extension unit 41 may be respectively provided with a speaker for outputting various notifications by audio. Also, the base unit 40 and the extension unit 41 may be respectively provided with a microphone, so as to be able to talk with the person being watched over via the information processing device 1 or the like. The information processing device 1 may thus be connected to equipment installed in the facility such as the nurse call system 4 or the like, via the external interface 15, and may perform various notifications in cooperation with that equipment.

Also, the information processing device 1 is connected to the nurse call via the external interface 15, as illustrated in FIG. 3. In this way, the information processing device 1, by being connected to equipment installed in the facility such as a nurse call via the external interface 15, performs notification for informing that there is an indication that the person being watched over is in impending danger, in cooperation with that equipment.

Note that the program 5 is a program for causing the information processing device 1 to execute processing that is included in operations discussed later, and corresponds to a “program” of the present invention. This program 5 may be recorded in the storage medium 6. The storage medium 6 is a medium that stores programs and other information by an electrical, magnetic, optical, mechanical or chemical action, such that the programs and other information are readable by a computer or other device, machine or the like. The storage medium 6 corresponds to a “storage medium” of the present invention. Note that FIG. 3 illustrates a disk-type storage medium such as a CD (Compact Disk) or a DVD (Digital Versatile Disk) as an example of the storage medium 6. However, the storage medium 6 is not limited to a disk-type storage medium, and may be a non-disk-type storage medium. Semiconductor memory such as flash memory can be given, for example, as a non-disk-type storage medium.

Also, for example, apart from a device exclusively designed for a service that is provided, a general-purpose device such as a PC (Personal Computer) or a tablet terminal may be used as the information processing device 1. Also, the information processing device 1 may be implemented using one or a plurality of computers.

### Exemplary Functional Configuration

Next, the functional configuration of the information processing device 1 will be described using FIG. 5. FIG. 5 illustrates the functional configuration of the information processing device 1 according to the present embodiment. The control unit 11 with which the information processing device 1 according to the present embodiment is provided expands the program 5 stored in the storage unit 12 in the RAM. The control unit 11 then controls the constituent elements by using the CPU to interpret and execute the program 5 expanded in the RAM. The information processing device 1 according to the present embodiment thereby functions as a computer that is provided with an image acquisition unit 21, a foreground extraction unit 22, a behavior detection unit 23, an anomaly determination unit 24, a notification unit 25, and a display control unit 26.

The image acquisition unit 21 acquires the captured image 3 captured by the camera 2. Depth information indicating the depth for each pixel measured by the depth sensor 8 is included in the captured image 3 that is acquired. The foreground extraction unit 22 extracts a foreground region of the captured image 3 from the difference between a background image set as the background of the captured image 3 and this captured image 3. The behavior detection unit 23 determines whether the positional relationship within real space between the target appearing in the foreground region and the bed satisfies a predetermined condition, based on the depth for each pixel within the foreground region that is indicated by the depth information. The behavior detection unit 23 then detects behavior of the person being watched over that is related to the bed, based on the result of the determination.

The anomaly determination unit 24 determines whether there is a possibility that an anomaly has occurred in the watching over by the watching system. The notification unit 25, in the case where it is determined that there is a possibility that an anomaly has occurred in the watching over by the watching system, then performs notification for informing that there is a possibility that watching over of the person being watched over is not being performed normally. The display control unit 26 controls screen display of the touch panel display 13.

For example, the anomaly determination unit 24 determines whether the state in which the depth for each pixel within the captured image 3 cannot be acquired by the depth sensor 8 has continued for a given period of time or longer. The notification unit 25, in the case where it is determined that the state in which the depth for each pixel within the captured image 3 cannot be acquired by the depth sensor 8 has continued for a given period of time or longer, performs notification for informing that there is a possibility that watching over of the person being watched over is not being performed normally. In this case, the display control unit 26 may perform screen display relating to this notification on the touch panel display 13.

Note that each function will be discussed in detail with an exemplary operation which will be discussed later. Here, in the present embodiment, an example will be described in which these functions are all realized by a general-purpose CPU. However, some or all of these functions may be realized by one or a plurality of dedicated processors. Also, in relationship to the functional configuration of the information processing device 1, functions may be omitted, replaced or added, as appropriate, according to the embodiment.

### 3. Exemplary Operation

Behavior Detection of Person being Watched Over

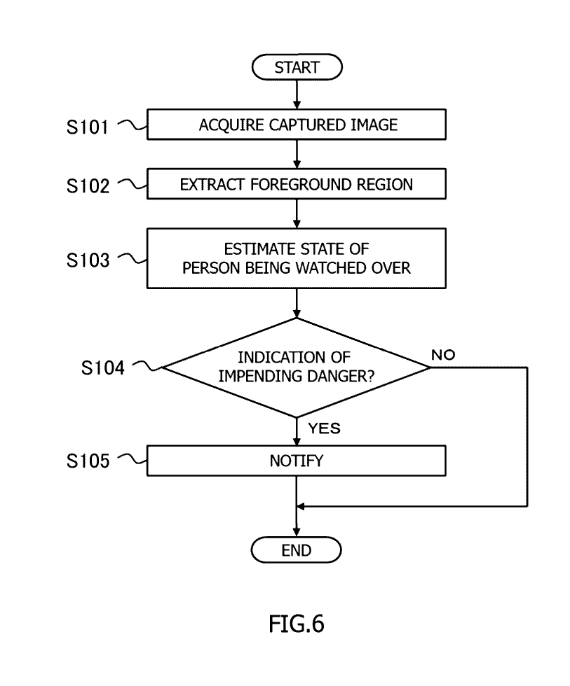

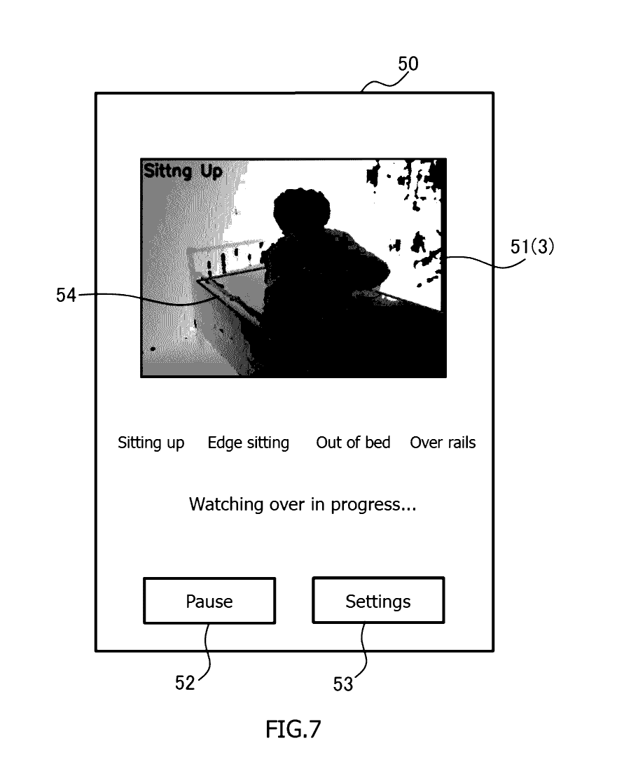

First, the processing procedure for detecting the behavior of the person being watched over by the information processing device 1 will be described using FIGS. 6 and 7. FIG. 6 illustrates a processing procedure relating to detection of the behavior of the person being watched over by the information processing device 1. FIG. 7 illustrates a screen 50 that is displayed on the touch panel display 13 when executing the processing relating to behavior detection.

The control unit 11 according to the present embodiment functions as the display control unit 26, and displays the screen 50 illustrated in FIG. 7 on the touch panel display 13, when performing watching over of the person being watched over using the processing procedure illustrated in FIG. 6. The screen 50 includes a region 51 for displaying captured images 3 that are being captured by the camera 2, a button 52 for accepting pausing of the watching processing illustrated in FIG. 6, and a button 53 for accepting various settings of the watching processing. The control unit 11 executes the processing of the following steps S101 to S105 while displaying a screen such as screen 50 on the touch panel display 13, and detects behavior of the person being watched over that is related to the bed. A user watches over the person being watched over, utilizing the result of this behavior detection.

Note that the processing procedure relating to the behavior detection described below is merely an example, and the respective processing may be modified to the full extent possible. With regard to the processing procedure described below, steps can be omitted, replaced or added, as appropriate, according to the embodiment. Also, the screen that is displayed on the touch panel display 13 when performing watching over of the person being watched over need not be limited to the screen 50 illustrated in FIG. 7, and may be set, as appropriate, according to the embodiment.

### Step S101

In step S101, the control unit 11 functions as the image acquisition unit 21, and acquires the captured image 3 captured by the camera 2. In the present embodiment, since the camera 2 is provided with the depth sensor 8, depth information indicating the depth for each pixel is included in the captured image 3 that is acquired. The control unit 11 acquires, as the captured image 3 including this depth information, a captured image 3 such as illustrated in FIGS. 2 and 7, for example, in which the gray value (pixel value) of each pixel is determined according to the depth for the pixel. In other words, the gray value of each pixel of the captured images 3 illustrated in FIGS. 2 and 7 corresponds to the depth of the target appearing in that pixel.

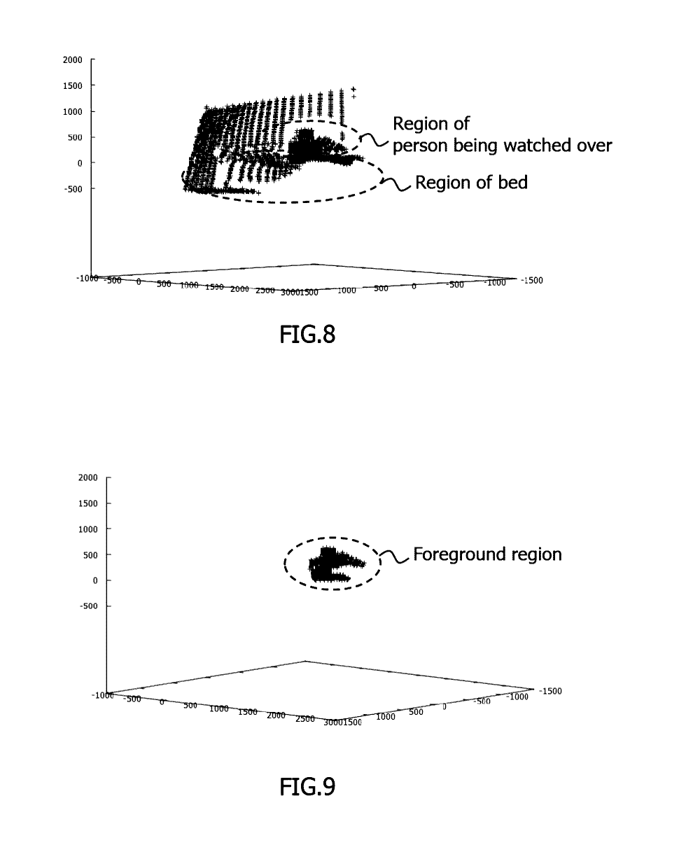

The control unit 11 is able to specify the position in real space of the target that appears in each pixel, based on the depth information, as described above. That is, the control unit 11 is able to specify, from the position (two-dimensional information) and depth for each pixel within the captured image 3, the position in three-dimensional space (real space) of the subject appearing within that pixel. For example, the state in real space of the subject appearing in the captured image 3 illustrated in FIG. 7 is illustrated in the following FIG. 8.

FIG. 8 illustrates the three-dimensional distribution of positions of the subject within the image capturing range that is specified based on the depth information that is included in the captured image 3. The three-dimensional distribution illustrated in FIG. 8 can be created by plotting each pixel within three-dimensional space with the position and depth within the captured image 3. In other words, the control unit 11 is able to recognize the state within real space of the subject appearing in the captured image 3, in a manner such as the three-dimensional distribution illustrated in FIG. 8.

Note that the information processing device 1 according to the present embodiment is utilized in order to watch over inpatients or facility residents in a medical facility or a nursing facility. In view of this, the control unit 11 may acquire the captured image 3 in synchronization with the video signal of the camera 2, so as to be able to watch over the behavior of inpatients or facility residents in real time. The control unit 11 may then immediately execute the processing of steps S102 to S105 discussed later on the captured image 3 that is acquired. The information processing device 1 realizes real-time image processing, by continuously executing such an operation without interruption, enabling the behavior of inpatients or facility residents to be watched over in real time.

### Step S102

Returning to FIG. 6, at step S102, the control unit 11 functions as the foreground extraction unit 22, and extracts a foreground region of the captured image 3, from the difference between a background image set as the background of the captured image 3 acquired at step S101 and the captured image 3. Here, the background image is data that is utilized in order to extract the foreground region, and is set to include the depth of a target serving as the background. The method of creating the background image may be set, as appropriate, according to the embodiment. For example, the control unit 11 may create the background image by calculating an average captured image for several frames that are obtained when watching over of the person being watched over is started. At this time, a background image including depth information is created as a result of the average captured image being calculated to also include depth information.

FIG. 9 illustrates the three-dimensional distribution of a foreground region, of the subject illustrated in FIGS. 7 and 8, that is extracted from the captured image 3. Specifically, FIG. 9 illustrates the three-dimensional distribution of the foreground region that is extracted when the person being watched over sits up in bed. The foreground region that is extracted utilizing a background image such as described above appears in a position that has changed from the state within real space shown in the background image. Thus, in the case where the person being watched over has moved in bed, the region in which the moving part of the person being watched over appears is extracted as this foreground region. For example, in FIG. 9, since the person being watched over has moved to raise his or her upper body (sit up) in bed, the region in which the upper body of the person being watched over appears is extracted as the foreground region. The control unit 11 determines the movement of the person being watched over, using such a foreground region.

Note that, in this step S102, the method by which the control unit 11 extracts the foreground region need not be limited to a method such as the above, and the background and the foreground may be separated using a background difference method, for example. As the background difference method, for example, a method of separating the background and the foreground from the difference between a background image such as described above and an input image (captured image 3), a method of separating the background and the foreground using three different images, and a method of separating the background and the foreground by applying a statistical model can be given. The method of extracting the foreground region is not particularly limited, and may be selected, as appropriate, according to the embodiment.

### Step S103

Returning to FIG. 6, in step S103, the control unit 11 functions as the behavior detection unit 23, and determines whether the positional relationship between the target appearing in the foreground region and the bed satisfies a predetermined condition, based on the depths of the pixels within the foreground region extracted in step S102. The control unit 11 then detects the behavior that the person being watched over is carrying out, based on the result of this determination.

Note that the method of detecting the behavior of the person being watched over, the predetermined condition for detecting each type of behavior, and the behavior to be detected are not particularly limited, and may be selected, as appropriate, according to the embodiment. Hereinafter, as an example of the method of detecting the behavior of the person being watched over, a method of detecting the person being watched over sitting up, being out of bed, edge sitting, and being over the rails based on the positional relationship between the bed upper surface and the foreground region will be described.

This bed upper surface is the surface on the upper side of the bed in the vertical direction, and is, for example, the upper surface of the bed mattress. The range within real space of this bed upper surface may be set in advance, may be set by analyzing the captured image 3 and specifying the position of the bed, or may be set as a result of the range being designated by the user within the captured image 3. Note that the reference for detecting the behavior of the person being watched over need not be limited to such a bed upper surface, and may be not only a physical target existing on the bed but a virtual target.

That is, the control unit 11 according to the present embodiment detects the behavior that the person being watched over is carrying out, based on the determination of whether the positional relationship within real space between the target appearing in the foreground region and the bed upper surface satisfies a predetermined condition. Thus, the predetermined condition for detecting the behavior of the person being watched over corresponds to a condition for determining whether the target appearing in the foreground region is included in a predetermined region (hereinafter, also referred to as the “detection region”) that is specified with the bed upper surface as a reference. In view of this, here, for convenience of description, a method of detecting the behavior of the person being watched over based on the relationship between this detection region and the foreground region will be described.

### (1) Sitting Up

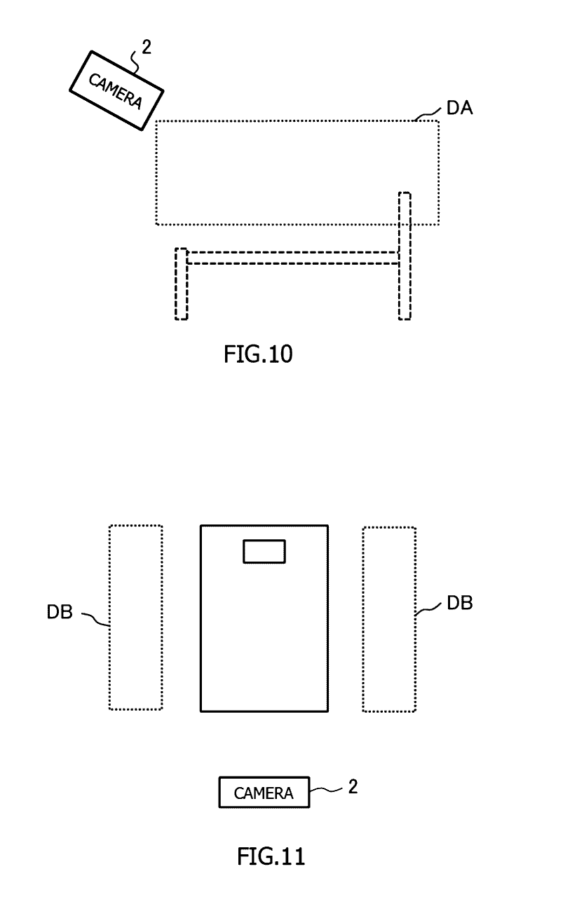

FIG. 10 schematically illustrates a detection region DA for detecting sitting up. In the case where the person being watched over sits up in bed, it is assumed that the foreground region illustrated in FIG. 9 will appear above the bed upper surface. Thus, the detection region DA for detecting sitting up may be set to a position that is a predetermined distance above the bed upper surface in the height direction of the bed, as illustrated in FIG. 10, for example. The range of the detection region DA is not particularly limited, and may be set, as appropriate, according to the embodiment. The control unit 11 may detect the person being watched over sitting up in bed, in the case where it is determined that the target appearing in the foreground region corresponding to a number of pixels greater than or equal to a threshold is included in the detection region DA.

### (2) Out of Bed

FIG. 11 schematically illustrates a detection region DB for detecting being out of bed. In the case where the person being watched over has gotten out of bed, it is assumed that the foreground region will appear in a position away from the side frame of the bed. Thus, the detection region DB for detecting being out of bed may be set to a position away from the bed upper surface in the width direction of the bed, as illustrated in FIG. 11, for example. The range of this detection region DB may be set, as appropriate, according to the embodiment, similarly to the detection region DA. The control unit 11 may detect the person being watched over being out of bed, in the case where it is determined that the target appearing in the foreground region corresponding to a number of pixels greater than or equal to a threshold is included in the detection region DB.

### (3) Edge Sitting

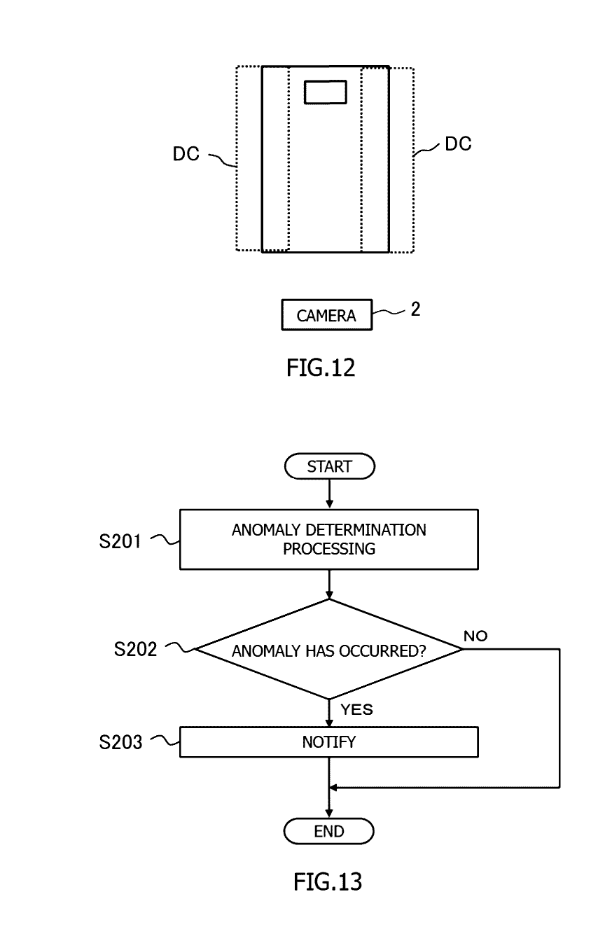

FIG. 12 schematically illustrates a detection region DC for detecting edge sitting. In the case where the person being watched over takes edge sitting on the bed, it is assumed that the foreground region will appear on the periphery of the side frame of the bed and also from above to below the bed. Thus, the detection region DC for detecting edge sitting may be set on the periphery of the side frame of the bed and also from above to below the bed, as illustrated in FIG. 12. The control unit 11 may detect the person being watched over edge sitting on the bed, in the case where it is determined that the target appearing in the foreground region corresponding to a number of pixels greater than or equal to a threshold is included in the detection region DC.

### (4) Over the Rails

In the case where the person being watched over leans out over the rails of the bed, or in other words, in the case where the person being watched over is positioned over the rails, it is assumed that the foreground region will appear on the periphery of the side frame of the bed and also above the bed. Thus, the detection region for detecting being over the rails may be set to the periphery of the side frame of the bed and also above the bed. The control unit 11 may detect the person being watched over being over the rails, in the case where it is determined that the target appearing in the foreground region corresponding to a number of pixels greater than or equal to a threshold is included in this detection region.

### (5) Other Processing

At this step S103, the control unit 11 performs detection of each type of behavior of the person being watched over in the manner described above. That is, the control unit 11 is able to detect the target behavior, in the case where it is determined that the above determination condition of the target behavior is satisfied. On the other hand, in the case where it is determined that the above determination condition of each type of behavior is not satisfied, the control unit 11 advances the processing to the next step S104, without detecting the behavior of the person being watched over.

Note that the method of detecting the behavior of the person being watched over need not be limited to the above method, and may be set, as appropriate, according to the embodiment. For example, the control unit 11 may calculate an average position of the foreground region, by taking the average position and depth of respective pixels within the captured image 3 that are extracted as the foreground region. The control unit 11 may then detect the behavior of the person being watched over, by determining whether the average position of the foreground region is included in the detection region set as a condition for detecting each type of behavior within real space.

Also, the control unit 11 may specify the part of the body appearing in the foreground region, based on the shape of the foreground region. The foreground region shows the change from the background image. Thus, the part of the body appearing in the foreground region corresponds to the moving part of the person being watched over. Based on this, the control unit 11 may detect the behavior of the person being watched over, based on the positional relationship between the specified body part (moving part) and the bed upper surface. Similarly to this, the control unit 11 may detect the behavior of the person being watched over, by determining whether the part of the body appearing in the foreground region that is included in the detection region for each type of behavior is a predetermined body part.

### Step S104

In step S104, the control unit 11 determines whether the behavior detected in step S103 is behavior showing an indication that the person being watched over is in impending danger. In the case where it is determined that the behavior detected in step S103 is behavior showing an indication that the person being watched over is in impending danger, the control unit 11 then advances the processing to step S105. On the other hand, in the case where the behavior of the person being watched over is not detected in step S103, or in the case where it is determined that the behavior detected in step S103 is not behavior showing an indication that the person being watched over is in impending danger, the control unit 11 ends the processing relating to this exemplary operation.

Behavior that is set as behavior showing an indication that the person being watched over is in impending danger may be selected, as appropriate, according to the embodiment. For example, as behavior that may possibly result in the person being watched over rolling or falling, assume that edge sitting is set as behavior showing an indication that the person being watched over is in impending danger. In this case, the control unit 11 determines that, when it is detected in step S103 that the person being watched over is edge sitting, the behavior detected in step S103 is behavior showing an indication that the person being watched over is in impending danger.

In the case of determining whether the behavior detected in this step S103 is behavior showing an indication that the person being watched over is in impending danger, the control unit 11 may utilize the transition in behavior of the person being watched over. For example, it is assumed that there is a greater chance of the person being watched over rolling or falling when changing from sitting up to edge sitting than when changing from being out of bed to edge sitting. In view of this, the control unit 11 may determine, in step S104, whether the behavior detected in step S103 is behavior showing an indication that the person being watched over is in impending danger in light of the transition in behavior of the person being watched over.

For example, assume that the control unit 11, when periodically detecting the behavior of the person being watched over, detects, in step S103, that the person being watched over has changed to edge sitting, after having detected that the person being watched over is sitting up. At this time, the control unit 11 may determine, in this step S104, that the behavior inferred in step S103 is behavior showing an indication that the person being watched over is in impending danger.

### Step S105

In step S105, the control unit 11 functions as the notification unit 25, and performs notification for informing that there is an indication that the person being watched over is in impeding danger. The method by which the control unit 11 performs the notification may be selected, as appropriate, according to the embodiment.

For example, the control unit 11 performs notification for informing that there is an indication that the person being watched over is in impending danger, in cooperation with equipment installed in the facility such as the nurse call system 4 that is connected to the information processing device 1. In the present embodiment, the control unit 11 may control the nurse call system 4 connected via the external interface 15 and perform a call by the nurse call system 4, as notification for informing that there is an indication that the person being watched over is in impending danger. It thereby becomes possible to appropriately inform the user who watches over the behavior of the person being watched over that there is an indication that the person being watched over is in impending danger.

Note that the control unit 11, as an example of the call by the nurse call system 4, causes at least one of the base unit 40 and the extension unit 41 to output predetermined audio, for example. This call may be performed with both or either one of the base unit 40 and the extension unit 41. The method of performing a call may be selected, as appropriate, according to the embodiment.

Also, for example, the control unit 11 may perform notification for informing that there is an indication that the person being watched over is in impending danger, by outputting predetermined audio from the speaker 14 that is connected to the information processing device 1. In the case where this speaker 14 is disposed in the vicinity of the bed, it is possible, by performing such notification with the speaker 14, to inform a person in the vicinity of the place where watching over is performed that there is an indication that the person being watched over is in impending danger. This person in the vicinity of the place where watching over is performed may include the person being watched over himself or herself. It is thereby possible to also report to the actual person being watched over that there is an indication that he or she is in impending danger.

Also, for example, the control unit 11 may cause a screen for informing that there is an indication that the person being watched over is in impending danger to be displayed on the touch panel display 13. Also, for example, the control unit 11 may perform such notification utilizing e-mail. In this case, an e-mail address of a user terminal serving as the notification destination may be registered in advance in the storage unit 12, and the control unit 11 may perform notification for informing that there is an indication that the person being watched over is in impending danger, utilizing this e-mail address registered in advance.

When this notification is completed, the control unit 11 ends the processing relating to this exemplary operation. The information processing device 1 may, however, periodically repeat the processing that is shown in an abovementioned exemplary operation, in the case of periodically detecting the behavior of the person being watched over. The interval for periodically repeating the processing may be set as appropriate. Also, the information processing device 1 may perform the processing shown in the above-mentioned exemplary operation, in response to a request from the user. Furthermore, the information processing device 1 may pause the processing relating to detecting the behavior of the person being watched over, in response to the operation of the button 52 provided on the screen 50.

As described above, the information processing device 1 according to the present embodiment detects the behavior of the person being watched over, by evaluating the positional relationship within real space between the moving part of the person being watched over and the bed, utilizing a foreground region and the depth of the subject. Thus, according to the present embodiment, behavior inference in real space that is in conformity with the state of the person being watched over is possible.

### Anomaly Determination

Next, processing for preventing the watching system from being left in a state in which watching over cannot be performed normally will be described using FIG. 13. FIG. 13 illustrates the processing procedure for preventing the watching system from being left in the state in which watching over, which is executed by the information processing device 1 according to the present embodiment, cannot be performed normally. Note that this processing relating to preventing the watching system from being left in an anomalous state may be executed at any timing, and may be executed periodically while the program 5 is being executed, for example. The processing procedure described below is merely an example, and the respective processing can be modified to the full extent possible. Also, with regard to the processing procedure that will be described below, steps can be replaced or added, as appropriate, according to the embodiment.

### Steps S201 and S202

In steps S201 and S202, the control unit 11 functions as the anomaly determination unit 24, and determines whether there is a possibility that an anomaly has occurred in the watching over by the watching system. The control unit 11, in the case where it is determined that there is a possibility that an anomaly has occurred in the watching over by the watching system, advances the processing to the next step S203. On the other hand, the control unit 11, in the case where it is determined that the occurrence of an anomaly in the watching over by the watching system is not possible, ends the processing relating to this exemplary operation. The method of determining whether there is a possibility that an anomaly has occurred in the watching over by the watching system may be selected as appropriate, according to the embodiment. Hereinafter, a specific method of determining whether there is a possibility that an anomaly has occurred in the watching over by the watching system is illustrated.

### (i) Non-Acquisition of Depth Information

The control unit 11 may, in the case where a state in which the depth for each pixel within the captured image 3 cannot be acquired by the depth sensor 8 continues for a given period of time or longer, for example, determine that there is a possibility that an anomaly has occurred in the watching over by the watching system. Various causes for not being able to acquire the depth corresponding to each pixel using the depth sensor 8 can be given. For example, in the case where a problem occurs in the depth sensor 8, the depth sensor 8 can no longer acquire the depth for each pixel. Also, if the depth sensor 8 is an infrared depth sensor, for example, the depth sensor 8 can no longer acquire the depth for each pixel in cases such as when an object that absorbs infrared light exists in the image capturing range, or when strong light such as sunlight is irradiated in the image capturing range.

In the case where the depth for each pixel cannot be acquired due to these causes, an error value is allocated to pixels whose depth cannot be acquired. The control unit 11 determines whether the state in which the depth for each pixel within the captured image 3 cannot be acquired by the depth sensor 8 has continued for a given period of time or longer, based on the duration that such error values continue to appear, for example. The predetermined time serving as a reference for determining that the above state has continued for a given period of time or longer may be determined in advance as a set value, may be determined using a value input by a user, or may be determined by being selected from a plurality of set values.

The control unit 11, in the case where it is determined that the state in which the depth for each pixel within the captured image 3 cannot be acquired by the depth sensor 8 has continued for a given period of time or longer, then evaluates that there is a possibility that an anomaly has occurred in the watching over by the watching system, and advances the processing to the next step S203. That is, the control unit 11 performs notification for informing that there is a possibility that the watching over by the watching system is not being performed normally, as will be discussed later. On the other hand, if this is not the case, the control unit 11 evaluates that there is not a possibility that an anomaly has occurred in the watching over by the watching system, and ends the processing relating to this exemplary operation.

As described above, the information processing device 1 evaluates the positional relationship within real space between the person being watched over and the bed, based on depth information. Thus, in the case where depth information cannot be acquired, the information processing device 1 can no longer detect the behavior of the person being watched over. In other words, the behavior of the person being watched over can no longer be watched over by the information processing device 1. In response to this, the information processing device 1 according to the present embodiment, in the case where it can be evaluated that depth information cannot be acquired, is able to report to a user or the like that there is a possibility that watching over of the person being watched over cannot be performed normally. Thus, according to the present embodiment, the watching system can be prevented from being left in a state in which there is a problem with the watching over due to not being able to acquire depth information.

Note that the control unit 11 may, in the case where the depth cannot be acquired for more than a predetermined proportion of the region of the bed, for example, determine that the state in which the depth for each pixel within the captured image 3 cannot be acquired by the depth sensor 8 has occurred. As described above, the information processing device 1 according to the present embodiment detects the behavior of the person being watched over, based on the positional relationship between the bed upper surface and the foreground region. Thus, if the depth information on the circumference of the bed can be acquired, the information processing device 1 is able to detect the behavior of the person being watched over.

In view of this, the control unit 11 according to the present embodiment measures the proportion of the bed region (e.g., bed upper surface) occupied by the region for which the depth cannot be acquired. The control unit 11, in the case where the proportion occupied by the region for which the depth cannot be acquired exceeds a predetermined value, may then determine that the state in which the depth for each pixel within the captured image 3 cannot be acquired by the depth sensor 8 has occurred. On the other hand, the control unit 11 may, in the case where the proportion occupied by the region for which the depth cannot be acquired is less than or equal to the predetermined value, determine that the state in which the depth for each pixel within the captured image 3 cannot be acquired by the depth sensor 8 has not occurred.

Determining that the state in which the depth for each pixel within the captured image 3 cannot be acquired has occurred, in the case where the depth cannot be acquired for a region that is unrelated to detecting the behavior of the person being watched over, can thereby be prevented. Thus, the possibility of the occurrence of an anomaly in the watching system being erroneously reported can be reduced. Note that the predetermined value serving as a reference for determining that the state in which depth information cannot acquire has occurred may be determined in advance as a set value, may be determined using a value input by a user, or may be determined by being selected from a plurality of set values.

### (ii) Shift in Image Capturing Range

Also, the control unit 11 may, in the case where a shift of a given amount or more has occurred in the image capturing range of the camera 2, for example, determine that there is a possibility that an anomaly has occurred in the watching over by the watching system. Various causes for a shift occurring in the image capturing range of the camera 2 can be given. For example, a shift occurs in the image capturing range of the camera 2 in the case where a person passing in proximity to the camera 2 bumps into the camera 2.

In the case where a shift of a given amount or more occurs in the image capturing range of the camera 2 due to such a cause, movement of a given amount or more occurs in the camera 2. Thus, the control unit 11 first detects an impact to the camera 2 based on the acceleration sensor 9. For example, the control unit 11 may detect an impact to the camera 2, in the case where the amount of movement of the camera 2 measured by the acceleration sensor 9 exceeds a predetermined value.

After detecting an impact to the camera 2 based on the acceleration sensor 9, the control unit 11 next compares the captured image 3 acquired before the impact is detected and the captured image 3 acquired after the impact is detected. The control unit 11 becomes able to acquire the captured image 3 before impact and the captured image 3 after impact from the storage unit 12, by causing the storage unit 12 to continue to hold the captured images 3 that are continuously acquired from the camera 2 for a given period of time, for example.

The method of comparing the captured image 3 before impact and the captured image 3 after impact may be selected as appropriate, according to the embodiment. The control unit 11 may compare these captured images 3, based on the degree of coincidence between the captured image 3 before impact and the captured image 3 after impact, for example. That is, the control unit 11 may determine whether a shift of a given amount or more has occurred in the image capturing range of the camera 2, based on the degree of coincidence between the captured image 3 before impact and the captured image 3 after impact.

In this case, the control unit 11 determines that a shift of a given amount or more has occurred in the image capturing range of the camera 2, when the degree of coincidence between the captured image 3 before impact and the captured image 3 after impact is less than or equal to a predetermined value. On the other hand, the control unit 11 determines that a shift of a given amount or more has not occurred in the image capturing range of the camera 2, when the degree of coincidence between the captured image 3 before impact and the captured image 3 after impact exceeds the predetermined value.

The control unit 11 then evaluates that there is a possibility that an anomaly has occurred in the watching over by the watching system, in the case where it is determined that a shift of a given amount or more has occurred in the image capturing range of the camera 2, and advances the processing to the next step S203. That is, the control unit 11 performs notification for informing that there is a possibility that the watching over by the watching system is not being performed normally, as will be discussed later. On the other hand, if this is not the case, the control unit 11 evaluates that there is not a possibility that an anomaly has occurred in the watching over by the watching system, and ends the processing relating to this exemplary operation.

As described above, the information processing device 1 detects behavior of the person being watched over that is related to the bed, as a result of the state in the vicinity of the bed appearing within the captured image 3. Thus, when the image capturing range of the camera 2 shifts, the state in the vicinity of the bed no longer sufficiently appears in the captured image 3, and the information processing device 1 may possibly be no longer able to detect the behavior of the person being watched over. In response to this, the information processing device 1 according to the present embodiment, in the case where it can be evaluated that the image capturing range has shifted by a given amount or more, reports to a user or the like that there is a possibility that watching over of the person being watched over cannot be performed normally. Thus, the watching system can be prevented from being left in a state in which there is a problem with the watching over that occurs due to a change in the orientation of the camera 2.

Note that the predetermined value serving as a reference for detecting an impact to the camera 2 and the predetermined value serving as a reference for determining a shift in the image capturing range of the camera 2 each may be determined in advance as a set value, may be determined using a value input by a user, or may be set by being selected from a plurality of set values.

(iii) Non-Recognition of Image Capturing Apparatus

Also, the control unit 11 may, in the case where the camera 2 cannot be recognized, for example, determine that there is a possibility that an anomaly has occurred in the watching over by the watching system. Various causes for the information processing device 1 becoming unable to recognize the camera 2 can be given. For example, the camera 2 can no longer be recognized by the information processing device 1 due to causes such as the wiring between the camera 2 and the information processing device 1 being cut and the power plug of the camera 2 being disconnected from the electrical socket.

In the case where the camera 2 cannot be recognized due to these causes, the control unit 11 can no longer access the camera 2 via the external interface 15. In view of this, the control unit 11 may determine whether the camera 2 can be recognized, based on whether the camera 2 can be accessed via the external interface 15.

The control unit 11, in the case where it is determined that the camera 2 cannot be recognized, then evaluates that there is a possibility that an anomaly has occurred in the watching over by the watching system, and advances the processing to the next step S203. That is, the control unit 11 performs notification for informing that there is a possibility that the watching over by the watching system is not being performed normally, as will be discussed later. On the other hand, if this is not the case, the control unit 11 evaluates that there is not a possibility that an anomaly has occurred in the watching over by the watching system, and ends the processing relating to this exemplary operation.

As described above, the information processing device 1 detects the behavior of the person being watched over by analyzing the captured image 3. Thus, when the camera 2 cannot be recognized, the information processing device 1 can no longer acquire the captured image 3 from the camera 2, and can no longer detect the behavior of the person being watched over. In response to this, the information processing device 1 according to the present embodiment, in the case where it can be evaluated that the captured image 3 cannot be acquired, reports to a user or the like that there is a possibility that watching over of the person being watched over cannot be performed normally. Thus, according to the present embodiment, the watching system can be prevented from being left in a state in which there is a problem with the watching over that occurs due to no longer being able to acquire the captured image 3.

### (iv) Non-Implementation of Behavior Detection

The control unit 11 may, in the case where behavior detection of the person being watched over according to step S103 has not been executed for a given period of time or longer, for example, determine that there is a possibility that an anomaly has occurred in the watching over by the watching system. Various causes for behavior detection of the person being watched over not being executed can be given. For example, behavior detection of the person being watched over is no longer be executed, due to causes such as the watching processing being left in a paused state due to operation of the button 52, or the setting screen being left in a displayed state due to operation of the button 53, as illustrated in FIG. 7.

In the case where the behavior detection processing is not performed due to these causes, the above paused state or the like will have been maintained. In view of this, the control unit 11 may determine whether detection of the behavior of the person being watched over has not been executed for a given period of time or longer, based on the period of time for which this paused state or the like has been maintained. Note that the predetermined period of time serving as a reference for determining whether the processing has not been executed for a given period of time or longer may be determined in advance as a set value, may be determined using a value input by a user, or may be determined by being selected from a plurality of set values.

The control unit 11, in the case where it is determined that detection of the behavior of the person being watched over not being executed for a given period of time or longer, then evaluates that there is a possibility that an anomaly has occurred in the watching over by the watching system, and advances the processing to the next step S203. That is, the control unit 11 performs notification for informing that there is a possibility that the watching over by the watching system is not being performed normally, as will be discussed later. On the other hand, if this is not the case, the control unit 11 evaluates that there is not a possibility that an anomaly has occurred in the watching over by the watching system, and ends the processing relating to this exemplary operation.

As described above, the information processing device 1 watches over the person being watched over, by detecting the behavior of the person being watched over. Thus, in the case where behavior detection of the person being watched over is not executed for a given period of time or longer, there is a possibility that the watching over of the person being watched over cannot be executed normally. In response to this, the information processing device 1 according to the present embodiment, in the case where it can be evaluated that the behavior of the person being watched over has not been detected for a given period of time or longer, reports to a user or the like that there is a possibility that watching over of the person being watched over is not being performed normally. Thus, according to the present embodiment, the watching system can be prevented from reporting in a state in which there is a problem with the watching over due to behavior detection of the person being watched over not being executed for a given period of time or longer.

### (v) Other Matters

Note that the state of the watching system in which it is determined that there is a possibility that an anomaly has occurred in the watching over by the watching system is not limited to the abovementioned example, and may be set as appropriate, according to the embodiment. For example, there is a possibility that processing for detecting the behavior of the person being watched over may not be performed appropriately, in the case where the load on the CPU of the information processing device (in the present embodiment, information processing device 1) that detects the behavior of the person being watched over is high. In view of this, the control unit 11 may, in the case where a given load or greater occurs on the CPU, determine that there is a possibility that watching over of the person being watched over cannot be performed normally.

The method of determining the load on the CPU may be selected as appropriate, according to the embodiment. For example, the control unit 11 is able to determine the load on the CPU, based on the usage rate of the CPU, the temperature of the CPU, and the like. The control unit 11 may, in the case where the usage rate of the CPU exceeds a predetermined value, determine that a given load or greater is occurring on the CPU. Also, the control unit 11 may, in the case where the temperature of the CPU exceeds a predetermined temperature, determine that a given load or greater is occurring on the CPU.

Also, for example, in the case where screen operation of the touch panel display 13 is locked by a password, there is a possibility that a user who erroneously inputs the password is not an authorized user of the watching system. There is a possibility that an unforeseen problem may occur in the watching system, in the case where the watching system is utilized by such a user. In view of this, the control unit 11 may, when a user erroneously inputs the password a predetermined number of times (e.g., 3 times), determine that there is a possibility that watching over of the person being watched over cannot be performed normally.

Note that, in these steps S201 and S202, the control unit 11 may determine whether there is a possibility that an anomaly has occurred in the watching over by the watching system, by employing one or a plurality of determination methods from the above determination methods. Also, the control unit 11 may accept selection of which determination methods to use from the above determination methods. The control unit 11 may then determine whether there is a possibility that an anomaly has occurred in the watching over by the watching system, utilizing the one or plurality of selected determination methods. In the case where it is determined that there is a possibility that an anomaly has occurred in the watching over by the watching system, the processing of the following step S203 is executed.

### Step S203

In step S203, the control unit 11 functions as the notification unit 25, and performs notification for informing that there is a possibility that watching over of the person being watched over is not being performed normally. The method of performing such notification may be selected as appropriate, according to the embodiment. For example, the control unit 11 may, as such notification informing that there is an indication of an anomaly having occurred, perform a call by the nurse call system 4, or may cause predetermined audio to be output from the speaker 14, similarly to the above notification that there is an indication of impending danger.

Also, for example, the control unit 11 may cause screen display in a predetermined mode to be performed on the touch panel display 13 as notification informing that there is an indication of an anomaly having occurred. As the predetermined mode of screen display, the control unit 11 may cause the screen that is displayed on the touch panel display 13 to flash, for example. Furthermore, the control unit 11 may, as notification informing that there is an indication of an anomaly having occurred, transmit an e-mail, or create a history recording the time at which indication of an anomaly having occurred was detected. Such a history of the indication of an anomaly having occurred can inform the time at which there was a possibility of watching over of the person being watched over not being performed normally to the user who operates the information processing device 1, after detecting an indication of an anomaly having occurred.

Note that the control unit 11 may utilize one or a plurality of devices in notification for informing that there is a possibility that watching over of the person being watched over is not being performed normally.

Also, the control unit 11 may perform notification for informing that there is a possibility that watching over of the person being watched over is not being performed normally, utilizing a specific device. In such a case, when the specific device for performing notification can no longer be recognized, the control unit 11 becomes unable to perform notification informing that there is an indication of an anomaly having occurred. In order to address this, the control unit 11 may, when the device for performing notification can no longer be recognized, perform this notification for informing that there is an indication of an anomaly having occurred, utilizing a different device from the device for performing notification.

For example, in the case where the nurse call system 4 is set as the device for performing notification for informing that there is an indication of an anomaly having occurred, the control unit 11, when controlling the nurse call system 4 normally, performs a call by the nurse call system 4 in response to the occurrence of the above situation.

On the other hand, when the nurse call system 4 can no longer be controlled normally, due to a reason such as the wiring 18 being cut, a call by the nurse call system 4 can no longer be performed. At this time, the control unit 11 may cause the speaker 14 to output predetermined audio, for example, as notification for informing that there is an indication of an anomaly having occurred, instead of the call by the nurse call system 4. The speaker 14 corresponds to an audio output device of the present invention. This predetermined audio is not particularly limited, and may, for example, be an audio message, a beep tone or the like that informs the content of the anomaly. It can thereby be audibly reported to a user or the like that an anomaly has occurred in the connection with the nurse call system 4, in addition to indication of the respective anomalous situations described above.

Also, the control unit 11 may, when a call by the nurse call system 4 cannot be performed, cause the touch panel display 13 to perform screen display in a predetermined mode, for example, as notification for informing that there is an indication of an anomaly having occurred, instead of the call by the nurse call system 4. The touch panel display 13 corresponds to a display device of the present invention. This predetermined mode of screen display is not particularly limited, and the control unit 11 may cause the screen that is displayed on the touch panel display 13 to flash, for example, as the predetermined mode of screen display. It can thereby be visually reported to a user or the like that an anomaly has occurred in the connection with the nurse call system 4, in addition to indication of the respective anomalous situations described above.

Instead of such a nurse call system 4, the device for performing notification for informing that there is an indication of an anomaly having occurred may be set in advance, or may be arbitrarily selected from devices that the control unit 11 recognizes when performing the notification. The occurrence of an anomaly in the watching system can thereby be reported, even in the case where a call by the nurse call system 4 cannot be performed normally.

### 4. Modifications

Although embodiments of the present invention have been described above in detail, the foregoing description is in all respects merely an illustration of the invention. It should also be understood that various improvement and modification can be made without departing from the scope of the invention.

### (1) Utilization of Area

For example, the image of the subject within the captured image 3 becomes smaller, the further the subject is from the camera 2, and the image of the subject within the captured image 3 increases, the closer the subject is to the camera 2. Although the depth of the subject appearing in the captured image 3 is acquired with respect to the surface of that subject, the area of the surface portion of the subject corresponding to each pixel of that captured image 3 does not necessarily coincide among the pixels.

In view of this, the control unit 11, in order to exclude the influence of the nearness or farness of the subject, may, in the above step S103, calculate the area within real space of the portion of the subject appearing in a foreground region that is included in the detection region. The control unit 11 may then detect the behavior of the person being watched over, based on the calculated area.

Note that the area within real space of each pixel within the captured image 3 can be derived as follows, based on the depth for the pixel. The control unit 11 is able to respectively calculate a length w in the lateral direction and a length h in the vertical direction within real space of an arbitrary point s (1 pixel) within the captured image 3 illustrated in FIG. 7, based on the following relational equations 1 and 2. Note that Ds indicates the depth at the point s. Vx indicates the field of view of the camera 2 in the lateral direction. Vy indicates the field of view of the camera 2 in the vertical direction. W indicates the number of pixels of the captured image 3 in the lateral direction. H indicates the number of pixels of the captured image 3 in the vertical direction. The coordinates of the central point (pixel) of the captured image 3 are set to (0, 0). The control unit 11 is able to acquire this information by accessing the camera 2, for example.

$$\begin{matrix}w = ( {D}_{s} \times tan \frac{{V}_{x}}{2} ) \text{/} \frac{W}{2} & ( 1 ) \\ h = ( {D}_{s} \times tan \frac{{V}_{y}}{2} ) \text{/} \frac{H}{2} & ( 2 )\end{matrix}$$

Accordingly, the control unit 11 is able to derive the area within real space of one pixel at a depth Ds, by the square of w, the square of h, or the product of w and h thus calculated. In view of this, the control unit 11, in the above step S103, calculates the total area within real space of those pixels in the foreground region that capture the target that is included in the detection region. The control unit 11 may then detect the behavior in bed of the person being watched over, by determining whether the calculated total area is included within a predetermine range. The accuracy with which the behavior of the person being watched over is detected can thereby be enhanced, by excluding the influence of the nearness or farness of the subject.

Note that this area may change greatly depending on factors such as noise in the depth information and the movement of objects other than the person being watched over. In order to address this, the control unit 11 may utilize the average area for several frames. Also, the control unit 11 may, in the case where the difference between the area of the region in the frame to be processed and the average area of that region for the past several frames before the frame to be processed exceeds a predetermined range, exclude that region from being processed.

### (2) Behavior Estimation Utilizing Area and Dispersion

In the case of detecting the behavior of the person being watched over utilizing an area such as the above, the range of the area serving as a condition for detecting behavior is set based on a predetermined part of the person being watched over that is assumed to be included in the detection region. This predetermined part may, for example, be the head, the shoulders or the like of the person being watched over. That is, the range of the area serving as a condition for detecting behavior is set, based on the area of a predetermined part of the person being watched over.

With only the area within real space of the target appearing in the foreground region, the control unit 11 is, however, not able to specify the shape of the target appearing in the foreground region. Thus, the control unit 11 may possibly erroneously detect the behavior of the person being watched over for the part of the body of the person being watched over that is included in the detection region. In view of this, the control unit 11 may prevent such erroneous detection, utilizing a dispersion showing the degree of spread within real space.

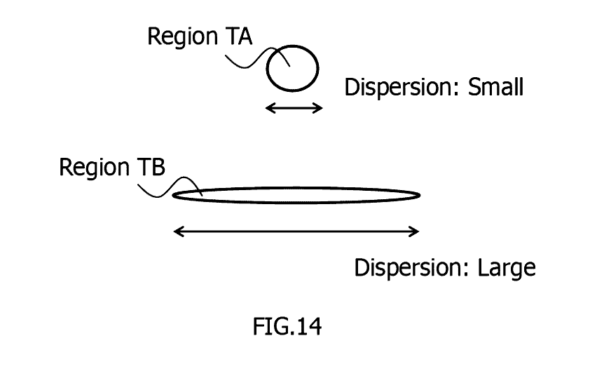

This dispersion will be described using FIG. 14. FIG. 14 illustrates the relationship between dispersion and the degree of spread of a region. Assume that a region TA and a region TB illustrated in FIG. 14 respectively have the same area. When inferring the behavior of the person being watched over with only areas such as the above, the control unit 11 recognizes the region TA and the region TB as being the same, and thus there is a possibility that the control unit 11 may erroneously detect the behavior of the person being watched over.

However, the spread within real space greatly differs between the region TA and the region TB, as illustrated in FIG. 14 (degree of horizontal spread in FIG. 14). In view of this, the control unit 11, in the above step S103, may calculate the dispersion of those pixels in the foreground region that capture the target included in the detection region. The control unit 11 may then detect the behavior of the person being watched over, based on the determination of whether the calculated dispersion is included in a predetermined range.

Note that, similarly to the example of the above area, the range of the dispersion serving as a condition for detecting behavior is set based on a predetermined part of the person being watched over that is assumed to be included in the detection region. For example, in the case where it is assumed that the predetermined part that is included in the detection region is the head, the value of the dispersion serving as a condition for detecting behavior is set in a comparatively small range of values. On the other hand, in the case where it is assumed that the predetermined part that is included in the detection region is the shoulder region, the value of the dispersion serving as a condition for detecting behavior is set in a comparatively large range of values.

### (3) Non-Utilization of Foreground Region

In the above embodiment, the control unit 11 (information processing device 1) detects the behavior of the person being watched over utilizing a foreground region that is extracted in step S102. However, the method of detecting the behavior of the person being watched over need not be limited to a method utilizing such a foreground region, and may be selected as appropriate according to the embodiment.

In the case of not utilizing a foreground region when detecting the behavior of the person being watched over, the control unit 11 may omit the processing of the above step S102. The control unit 11 may then function as the behavior detection unit 23, and detect behavior of the person being watched over that is related to the bed, by determining whether the positional relationship within real space between the bed reference plane and the person being watched over satisfies a predetermined condition, based on the depth for each pixel within the captured image 3. As an example of this, the control unit 11 may, as the processing of step S103, analyze the captured image 3 by pattern detection, graphic element detection or the like to specify an image that is related to the person being watched over, for example. This image related to the person being watched over may be an image of the whole body of the person being watched over, and may be an image of one or a plurality of body parts such as the head and the shoulders. The control unit 11 may then detect behavior of the person being watched over that is related to the bed, based on the positional relationship within real space between the specified image related to the person being watched over and the bed.

Note that, as described above, the processing for extracting the foreground region is merely processing for calculating the difference between the captured image 3 and the background image. Thus, in the case of detecting the behavior of the person being watched over utilizing the foreground region as in the above embodiment, the control unit 11 (information processing device 1) becomes able to detect the behavior of the person being watched over, without utilizing advanced image processing. It thereby becomes possible to accelerate processing relating to detecting the behavior of the person being watched over.

### REFERENCE SIGNS LIST

1 Information processing device
2 Camera
3 Captured image
4 Nurse call system
5 Program
6 Storage medium
8 Depth sensor
9 Acceleration sensor
11 Control unit
12 Storage unit
13 Touch panel display
14 Speaker
15 External interface
16 Communication interface
17 Drive
21 Image acquisition unit
22 Foreground extraction unit
23 Behavior detection unit
24 Anomaly determination unit
25 Notification unit
26 Display control unit

1. An information processing device comprising:
an image acquisition unit configured to acquire a captured image captured by an image capturing device that is installed in order to watch for behavior, in a bed, of a person being watched over and includes a depth sensor configured to measure a depth of a subject, the captured image including depth information indicating the depth for each pixel within the captured image that is measured by the depth sensor;
a behavior detection unit configured to detect behavior, related to the bed, of the person being watched over, by determining whether a positional relationship within real space between the person being watched over and a region of the bed satisfies a predetermined condition, based on the depth for each pixel within the captured image that is indicated by the depth information;
an anomaly determination unit configured to determine whether a state in which the depth for each pixel within the captured image cannot be acquired by the depth sensor has continued for a given period of time or longer; and
a notification unit configured to perform, in a case where it is determined that the state in which the depth for each pixel within the captured image cannot be acquired by the depth sensor has continued for a given period of time or longer, notification for informing that there is a possibility that watching over of the person being watched over is not being performed normally.

2. The information processing device according to claim 1,
wherein the anomaly determination unit determines, in a case where the depth cannot be acquired for more than a predetermined proportion of the region of the bed, that the state in which the depth for each pixel within the captured image cannot be acquired by the depth sensor has occurred.

3. The information processing device according to claim 1, further comprising:
a foreground extraction unit configured to extract a foreground region of the captured image from a difference between the captured image and a background image set as a background of the captured image,
wherein the behavior detection unit detects the behavior, related to the bed, of the person being watched over, by determining whether the positional relationship within real space between the person being watched over and the region of the bed satisfies a predetermined condition, utilizing, as a position of the person being watched over, a position within real space of a target appearing in the foreground region that is specified based on the depth for each pixel within the foreground region.

4. The information processing device according to claim 1,
wherein the image capturing device further includes an acceleration sensor,
the anomaly determination unit determines, after detecting an impact to the image capturing device based on the acceleration sensor, whether a shift of a given amount or more has occurred in an image capturing range of the image capturing device, by comparing the captured image prior to the impact and the captured image after the impact, and
the notification unit performs, in a case where it is determined that a shift of a given amount or more has occurred in the image capturing range of the image capturing device, notification for informing that there is a possibility that watching over of the person being watched over is not being performed normally.

5. The information processing device according to claim 1,
wherein the information processing device is connected to a nurse call system for calling a person who watches over the person being watched over, and
the notification unit performs a call by the nurse call system, as the notification for informing that there is a possibility that watching over of the person being watched over is not being performed normally.

6. The information processing device according to claim 5,
wherein the information processing device is connected to an audio output device for outputting audio, and
the notification unit causes, in a case where the call by the nurse call system cannot be performed, the audio output device to perform output of predetermined audio, as the notification for informing that there is a possibility that watching over of the person being watched over is not being performed normally, instead of the call by the nurse call system.

7. The information processing device according to claim 5,
wherein the information processing device is connected to a display device for performing screen display, and
the notification unit causes, in a case where the call by the nurse call system cannot be performed, the display device to perform screen display in a predetermined mode, as the notification for informing that there is a possibility that watching over of the person being watched over is not being performed normally, instead of the call by the nurse call system.

8. The information processing device according to claim 1,
wherein the anomaly determination unit determines whether the information processing device recognizes the image capturing device, and
the notification unit performs, in a case where it is determined that the information processing device does not recognize the image capturing device, notification for informing that there is a possibility that watching over of the person being watched over is not being performed normally.

9. The information processing device according to claim 1,
wherein the anomaly determination unit determines whether detection of the behavior of the person being watched over by the behavior detection unit has not been executed for a given period of time or longer, and
the notification unit performs, in a case where it is determined that detection of the behavior of the person being watched over by the behavior detection unit has not been executed for a given period of time or longer, notification for informing that there is a possibility that watching over of the person being watched over is not being performed normally.

10. The information processing device according to claim 1,
wherein the depth sensor that is included in the image capturing device is an infrared depth sensor that measures depth based on infrared irradiation.

11. An information processing method in which a computer executes:
a step of acquiring a captured image captured by an image capturing device that is installed in order to watch for behavior, in a bed, of a person being watched over and includes a depth sensor configured to measure a depth of a subject, the captured image including depth information indicating the depth for each pixel within the captured image that is measured by the depth sensor;
a step of detecting behavior, related to the bed, of the person being watched over, by determining whether a positional relationship within real space between the person being watched over and a region of the bed satisfies a predetermined condition, based on the depth for each pixel within the captured image that is indicated by the depth information;
a step of determining whether a state in which the depth for each pixel within the captured image cannot be acquired by the depth sensor has continued for a given period of time or longer, and
a step of performing, in a case where it is determined that the state in which the depth for each pixel within the captured image cannot be acquired by the depth sensor has continued for a given period of time or longer, notification for informing that there is a possibility that watching over of the person being watched over is not being performed normally.

12. A non-transitory recording medium recording a program to cause a computer to execute:
a step of acquiring a captured image captured by an image capturing device that is installed in order to watch for behavior, in a bed, of a person being watched over and includes a depth sensor configured to measure a depth of a subject, the captured image including depth information indicating the depth for each pixel within the captured image that is measured by the depth sensor;
a step of detecting behavior, related to the bed, of the person being watched over, by determining whether a positional relationship within real space between the person being watched over and a region of the bed satisfies a predetermined condition, based on the depth for each pixel within the captured image that is indicated by the depth information;
a step of determining whether a state in which the depth for each pixel within the captured image cannot be acquired by the depth sensor has continued for a given period of time or longer; and
a step of performing, in a case where it is determined that the state in which the depth for each pixel within the captured image cannot be acquired by the depth sensor has continued for a given period of time or longer, notification for informing that there is a possibility that watching over of the person being watched over is not being performed normally.
